(19) United States
(12) Patent Application Publication
(10) Pub. No.: US 2026/0232332 A1
(43) Pub. Date: Aug. 13, 2026

(54) EMBOLIC PROTECTION DEVICE

(71) Applicant: MicroVention, Inc., Aliso Viejo, CA (US)

(72) Inventor: Belete Degaga, Aliso Viejo, CA (US)

(73) Assignee: MicroVention, Inc., Aliso Viejo, CA (US)

(21) Appl. No.: 19/150,523

(22) PCT Filed: Jan. 31, 2024
(86) PCT No.: PCT/US2024/013828

§ 371 Date: Jul. 23, 2025

Related U.S. Application Data
(60) Provisional application No. 63/482,482, filed on Jan. 31, 2023.

(51) Int. Cl. A61B 17/22; A61B 17/00

(52) U.S. Cl. CPC A61B 17/22; A61B 17/00234; A61B 2017/00238; A61B 2017/00305; A61B 2017/22001; A61B 2017/22065

(57) ABSTRACT

An embolic protection device may include an inflatable embolic balloon with one or more embolic capture elements located at or around its outer surface. The balloon may be inflatable so as to move the one or more embolic capture elements near or against the wall of a vessel. Emboli, and particularly emboli that are relatively more diffuse, softer, more friable, contain more inflammatory cells, and/or may lack fibrous caps, are captured or stopped by the one or more embolic capture elements as blood passes through the one or more embolic capture elements.

### RELATED APPLICATIONS

This application claims benefit of and priority to U.S. Provisional Application Ser. No. 63/482,482 filed Jan. 31, 2023 entitled Embolic Protection Device, which is hereby incorporated herein by reference in its entirety.

### BACKGROUND

Certain procedures within a patient's vascular system may risk dislodging emboli or similar material. Emboli may include blood clots, fat, bacteria, or other material. Once dislodged, the emboli may travel through the patient's blood stream and lodge within a smaller vessel, blocking some or all of the blood flow at that location. Depending on where the emboli lodges, it may cause a variety of damage to different areas of the body, including vital organs such as the brain or lungs.

The treatment of atherosclerosis is one specific example that may risk dislodging emboli. Atherosclerosis typically results from a buildup of plaque composed of fats, cholesterol, and/or similar substances in and on artery walls, resulting in a narrowing or blockage of the artery. Common treatment procedures involve expanding an angioplasty balloon against the plaque and/or expanding a stent against the plaque.

More recently, embolic protection devices have been deployed downstream of a treatment location to help collect and remove any emboli or other material that has been dislodged during a procedure.

### SUMMARY

In some aspects, the techniques described herein relate to an embolic protection device, including: an elongated catheter body; an embolic balloon located at a distal portion of the elongated catheter body; and, one or more embolic capture elements that are each positioned around the embolic balloon; wherein the one or more embolic capture elements extend radially outward from the embolic balloon when the embolic balloon is inflated to capture embolic material.

In some aspects, the techniques described herein relate to an embolic protection device, wherein the one or more embolic capture elements include 2, 3, 4, 5, 6, 7, 8, 9, or 10 embolic capture elements.

In some aspects, the techniques described herein relate to an embolic protection device, wherein embolic capture elements have a uniform spacing or a non-uniform spacing.

In some aspects, the techniques described herein relate to an embolic protection device, wherein the one or more embolic capture elements form a conical shape opening towards a proximal end of the elongated catheter body.

In some aspects, the techniques described herein relate to an embolic protection device, wherein the one or more embolic capture elements form a flat disc shape around the embolic balloon.

In some aspects, the techniques described herein relate to an embolic protection device, wherein the one or more embolic capture elements form a wave-like shape that progressively increases and decreases in radius along a longitudinal length of the embolic balloon.

In some aspects, the techniques described herein relate to an embolic protection device, wherein the one or more embolic capture elements include a wire forming a coil shape around the embolic balloon.

In some aspects, the techniques described herein relate to an embolic protection device, wherein the one or more embolic capture elements include a plurality of struts connected together to form a web surrounding the embolic balloon.

In some aspects, the techniques described herein relate to an embolic protection device, wherein the one or more embolic capture elements include a plurality of braided wires that form a mesh surrounding the embolic balloon.

In some aspects, the techniques described herein relate to an embolic protection device, wherein the one or more embolic capture elements are connected to an outer surface of the embolic balloon via an elastomeric band or metal coil ring.

In some aspects, the techniques described herein relate to an embolic protection device, wherein the one or more embolic capture elements are embedded within a wall of the embolic balloon.

In some aspects, the techniques described herein relate to an embolic protection device, further including a framework connected to the one or more embolic capture elements and to the elongated catheter body, supporting the embolic capture elements separately of the embolic balloon.

In some aspects, the techniques described herein relate to an embolic protection device, wherein the framework is connected to the elongated catheter body distally, proximally, or both distally and proximally of the embolic balloon.

In some aspects, the techniques described herein relate to an embolic protection device, further including a stent that is deployable from the elongated catheter body.

In some aspects, the techniques described herein relate to an embolic protection device, further including an angioplasty balloon connected on the elongated catheter body.

In some aspects, the techniques described herein relate to an embolic protection device, further including a filter material having a plurality of pores allowing blood to pass therethrough.

In some aspects, the techniques described herein relate to an embolic protection device, including: an elongated catheter body; an embolic balloon located at a distal portion of the elongated catheter body; the embolic balloon having an inflated state and a deflated state; and, one or more embolic capture elements that are each positioned around the embolic balloon; wherein the one or more embolic capture elements extend radially away from the embolic balloon when the embolic balloon is in the inflated state.

In some aspects, the techniques described herein relate to an embolic protection device, wherein the one or more embolic capture elements each include one or more wires forming a coil surrounding the embolic balloon.

In some aspects, the techniques described herein relate to an embolic protection device, wherein each coil further includes a blood permeable membrane connected to a proximal side or a distal side of the coil.

In some aspects, the techniques described herein relate to an embolic protection device, including: an elongated catheter body; an embolic balloon located at a distal portion of the elongated catheter body; and, one or more embolic capture means for capturing embolic material; wherein each are each positioned around the embolic balloon.

In some aspects, the techniques described herein relate to a method of using an embolic protection device, including: advancing a distal portion of an elongated catheter body near a treatment location; inflating an embolic balloon located at a distal portion of the elongated catheter body such that one or more embolic capture elements extend radially from the embolic balloon; and, performing a treatment at the treatment location such that embolic material is captured by the embolic capture elements.

In some aspects, the techniques described herein relate to a method, wherein performing the treatment further includes inflating an angioplasty balloon and/or delivery a stent.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 is a side view of an embolic protection device according to one example.

FIG. 2 is an enlarged view of the embolic protection device of FIG. 1.

FIG. 3 is a perspective view of the embolic protection device of FIG. 1.

FIG. 4 is a side view of the embolic protection device of FIG. 1 in an uninflated configuration.

FIG. 5 is a perspective view of an embolic protection device with a filter material according to one example.

FIG. 6 is a cross sectional view of an embolic capture element along the cross sectional lines of FIG. 5 according to one example.

FIG. 7 is a cross sectional view of an embolic capture element along the cross section lines of FIG. 2 according to one example.

FIG. 8A is a side view of an embolic capture element according to one example.

FIG. 8B is a cross sectional view of an embolic capture element along the cross section lines of FIG. 8A according to one example.

FIG. 9A is a side view of an embolic capture element according to one example.

FIG. 9B is a cross sectional view of an embolic capture element along the cross section lines of FIG. 9A according to one example.

FIG. 10 is a side view of an embolic capture element according to one example.

FIG. 11 is a side view of an embolic protection device with a stent according to one example.

FIG. 12 is a side view of an embolic protection device with an angioplasty balloon according to one example.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Embolic protection devices are typically deployed downstream of a desired treatment location to help collect any emboli or other material dislodged during a treatment procedure. For example, an embolic protection device may be deployed downstream of a plaque in a vessel and then an angioplasty balloon may be expanded against the plaque, or a stent may be deployed against the plaque. Present embolic protection devices are typically conical, mesh structures in their expanded states which allow material to be captured within the mesh cone and then withdrawn from the patient.

The composition, density, and texture of different plaques may vary, particularly based on the plaque location. While some plaques may be relatively large, solid, and dense, others (e.g., in connection with saphenous vein grafts) may be relatively more diffuse, softer, more friable, contain more inflammatory cells, and/or may lack fibrous caps. These softer plaques may have more extensive thrombotic burden and distal embolization during percutaneous treatment, often resulting in microvascular disfunction, the “no-reflow” phenomenon, and periprocedural myocardial infarction.

The present specification is generally directed to an embolic protection device that may better capture certain types of disrupted emboli and similar material from plaques, such as those that are relatively more diffuse, softer, more friable, contain more inflammatory cells, and/or may lack fibrous caps. However, the present specification may be used with other types of plaques as well.

An example embolic protection device may generally include an inflatable embolic balloon with one or more embolic capture elements located at or around its outer surface. The balloon may be inflatable so as to move the one or more embolic capture elements near or against the wall of a vessel. Emboli, and particularly emboli that are relatively more diffuse, softer, more friable, contain more inflammatory cells, and/or may lack fibrous caps, may be captured or stopped by the one or more embolic capture elements as blood passes through the one or more embolic capture elements.

With the embolic balloon inflated, less blood may otherwise flow through the vessel. However, due to turbulence flow the blood that does pass around the embolic balloon may do so at a higher speed in different directions which may allow certain types of emboli to be better captured. This may better move certain types of material described above towards the embolic capture elements for capture. Put another way, the embolic balloon may create blood flow patterns and conditions that better capture certain embolic material within a vessel.

The embolic balloon and the embolic capture elements may be located on a distal portion of an elongated element, such as a catheter or tube, which has a passage opening at a proximal location and within the embolic balloon to allow selective inflation or deflation. Inflation may typically mean delivering a material, such as fluid (e.g., saline and/or contrast) inside of the embolic balloon so that it increases in size. Deflation may typically mean removing the delivered material from the embolic balloon so that it decreases in size. The balloon and the embolic capture elements may be the only treatment component on the distal portion of the elongated element or other treatment components may also be included. For example, an angioplasty balloon (with its own inflation lumen) may also be connected proximally of the embolic balloon and/or a stent may also be positioned proximal or distal of the embolic balloon. Alternatively, the angioplasty balloon, stent, or other treatment device may be included on a separate treatment catheter.

The embolic balloon may be composed of a compliant balloon material, semi-compliant balloon material, or a non-compliant balloon material, any of which may be covered with biocompatible material (e.g., ePTFE, dacron, etc.). The embolic balloon may have an inflated state or size that is close to or smaller than that of a target vessel. In one example, the embolic balloon may have an inflated diameter within an inclusive range of about 1 mm to about 25 mm. In some examples, the embolic balloon may have an inflated diameter having a uniform diameter or different diameters. In some examples, when the embolic balloon is inflated, the balloon may have a shape that conforms to the curvatures of the vessel wall. The embolic balloon may also have a deflated state or size that fits within a delivery sheath or catheter.

The embolic capture elements may have a variety of different shapes when the embolic balloon is inflated. For example, the embolic capture elements may form a shape that increases in diameter in one direction (e.g., in a proximal direction or a distal direction) and around the perimeter of the embolic balloon, similar to a cone or concave shape. In another example, the embolic capture elements may extend in a generally perpendicular angle relative to an axis of the embolic balloon. In another example, the embolic capture elements may form a plurality of waves along the length of embolic balloon. The embolic balloon may include 1, 2, 3, 4, 5, 6, 7, 8, 9, 10, or more embolic capture elements.

In one example, the embolic capture elements may be formed from one or more wires coiled at least once around the outside or externally of the embolic balloon, and in another example, coiled a plurality of times around the embolic balloon to form a coil. The one or more wire coils may be uncovered or may be covered with a layer of material having a porosity that is blood permeable (e.g., blood-permeable ePTFE). In another example, the embolic capture elements may be formed as a circular strut or mesh framework surrounding the outside of the embolic balloon. In some examples, the embolic capture elements may be attached to an outer surface of the balloon (e.g., via a polymer or elastomeric band or a metal or polymer coil ring) or partially embedded within the balloon material. Alternatively, the embolic capture elements may be connected to proximal and/or distal locations relative to the balloon of the underlying catheter body (e.g., a wire framework separate of the balloon). The layer of material may cover one side of the one or more wire coils, struts, or mesh framework, or may cover both sides of these structures (e.g., proximal side and/or distal side).

In some examples, one or more wires, struts, and/or framework of the embolic capture elements may be composed of a shape memory material, such as Nitinol, and the “memorized” or heat set shape may be any of the shapes described in this specification. In some examples, the one or more wires, struts, and/or framework of the embolic capture elements may be composed of a resilient or elastic polymer that may similarly have a “memorized” shape that it returns to when unconstrained.

In some examples, if the embolic capture elements are composed of one or more wire coils, the one or more wire coils may longitudinally expand and foreshorten in length between their radially expanded configuration and radially compressed configuration. The one or more wire coils may also helically rotate as they transition between their radially expanded configuration and radially compressed configuration. As the embolic balloon expands or contracts, it may apply force on the embolic capture elements (e.g., via an elastomeric band attached to both the embolic balloon and an embolic capture element). For example, the embolic balloon may radially push or radially pull one end of an embolic capture element, causing it to radially expand or radially contract. In some examples, the outer sheath or delivery catheter that the embolic protection device is deployed from may have a conical or funnel-shaped distal end of its lumen. This conical or funnel-shape may help force the embolic capture elements into a radially contracted configuration of sufficient size to fit within the remaining portion of the lumen of the sheath or delivery catheter.

When the embolic balloon is expanded, the embolic capture elements may radially expand, for example within an inclusive range of about 2 mm to about 15 mm, depending on the target location. Since an aorta may have an internal diameter of about an inch (25.4 mm) for example and some of the smallest arteries may have an internal diameter between about 3 to 5 mm, the total radial dimensions of the embolic balloon may vary widely depending on the intended target location, such as an inflated diameter within an inclusive range of about 1 mm to about 25 mm.

In operation, a distal portion of the embolic protection device may be positioned downstream of a target treatment location (e.g., a plaque) and its embolic balloon inflated such that its embolic capture elements may be in contact with or near contact with the walls of the vessel. Next, a treatment device, such as an angioplasty balloon or stent (either separate catheters or part of the embolic protection device) may be positioned at the target treatment location and actuated. Any dislodged emboli or other material moves along with the blood flow in the vessel, around the proximal portion of the balloon, and against the embolic capture elements. Finally, the embolic protection device, including the captured emboli/material can be deflated and removed from the patient.

With regard to example of the embolic capture elements that comprise one or more wires that form a coil with a plurality of loops, these loops may radially expand and contract in different ways. For example, as the loops of the coils decrease in diameter, they may create further loops along the deflated balloon, effectively increasing the longitudinal size of the embolic capture element (and vise-versa for expansion). Alternatively, the loops of the coils may spread out from each other during radial contraction without creating additional loops but simply increasing a gap size between the loops to accommodate the reduction in diameter (e.g., simply stretching a coil verse winding or twisting a coil). Alternatively, the loops of the coil may be bent or otherwise deformed to fit within the lumen of a sheath or delivery catheter. If a membrane is attached to the loops of these coils, it may be arranged such that there is sufficient slack between loops of the coils to accommodate any gap size changes or twisting of the membrane that may occur under any of these configurations.

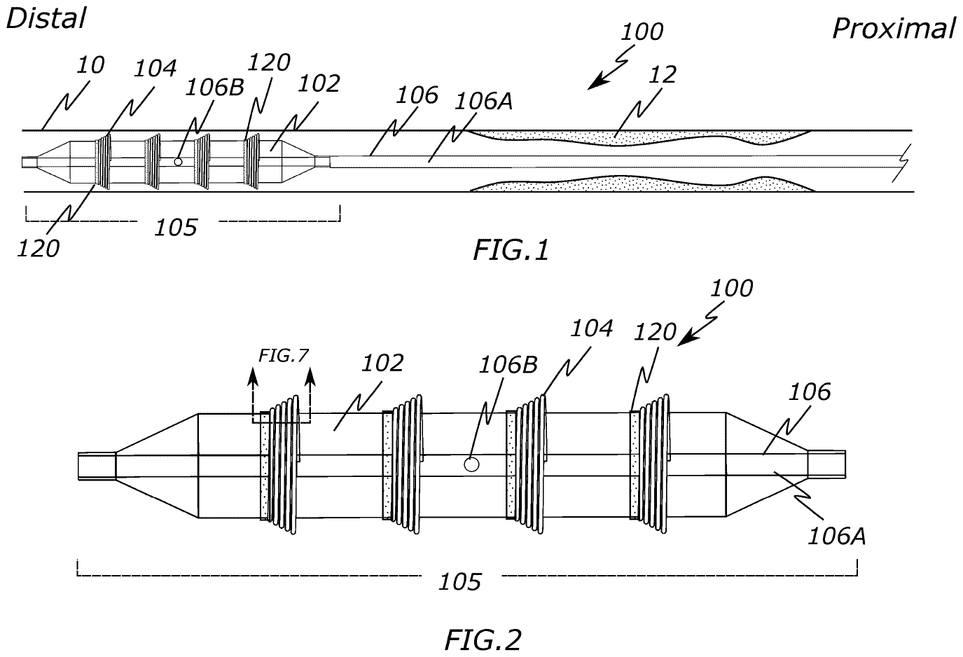

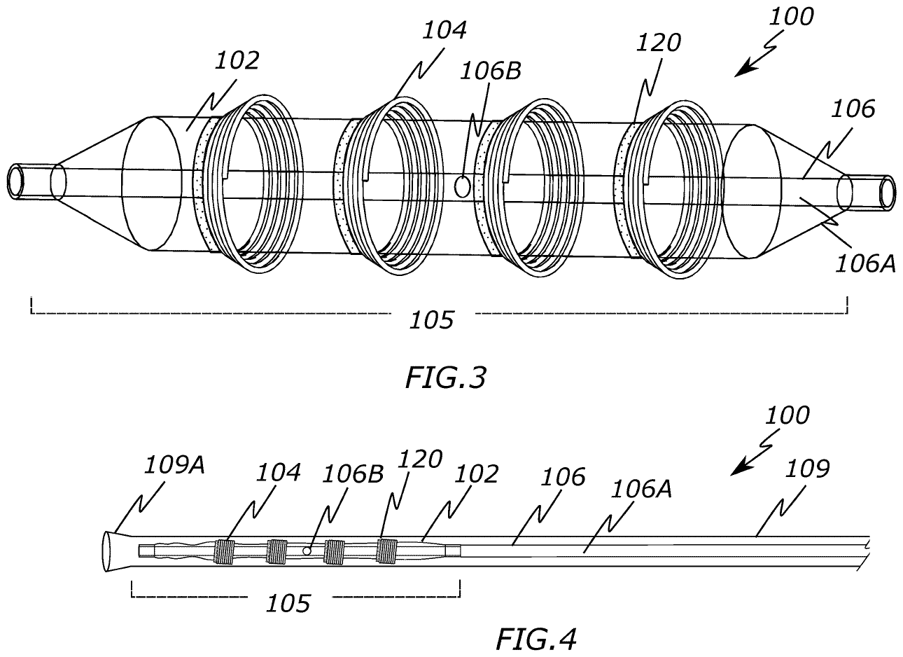

FIGS. 1-4 illustrate an example of an embolic protection device 100. In particular, FIG. 1 illustrates a side view of the embolic protection device 100 within a vessel 10 containing plaque 12, FIG. 2 illustrates a magnified view of a distal portion of the embolic protection device 100, FIG. 3 illustrates a perspective view of the distal portion of the embolic protection device 100, and FIG. 4 illustrates a view of the embolic protection device 100 in a deflated configuration or state. These figures will be discussed concurrently.

The embolic protection device 100 may comprise an elongated catheter body 106 having a distal portion 105 on which an embolic balloon 102 and one or more embolic capture elements 104 may be located. When the embolic balloon 102 is inflated, it may help direct blood flow, including embolic material, towards the one or more embolic capture elements 104 which may capture the embolic material so that it may be removed.

The elongated catheter body 106 may include an inflation lumen 106A opening at or near a proximal end of the elongated catheter body 106, such as at a catheter hub with one or more openings. The elongated catheter body 106 may also include one or more inflation openings 106B located in the distal portion 105 so that the inflation lumen 106A is in fluid communication with an interior of the embolic balloon 102. As inflation media, such as saline and/or contrast, is injected into the inflation lumen 106A from a proximal end of the elongated catheter body 106, it travels distally through the inflation lumen 106A and out the one or more inflation openings 106B to increase the diameter of the embolic balloon 102. The inflation media may be withdrawn from the embolic balloon 102 along the same pathway to deflate the embolic balloon 102.

The embolic balloon 102 may be composed of a compliant balloon material (e.g., elastic material), semi-compliant balloon material, or a non-compliant balloon material, any of which may be covered with biocompatible material (e.g., ePTFE, dacron, etc.). A non-compliant balloon material may be particularly helpful for limiting the expanded diameter of the embolic balloon 102. While a single embolic balloon 102 is disclosed, it is also contemplated that several balloons may also be possible. While the embolic balloon 102 is depicted as having a generally uniform shape in its expanded/inflated configuration, the embolic balloon 102 may alternatively have an expanded/inflated configuration with regions of multiple diameters (e.g., a larger diameter region and a smaller diameter region).

In some examples, one or more embolic capture elements 104 may be located on an outer surface, perimeter, or circumference of the embolic balloon 102. As the embolic balloon 102 inflates and radially expands, the embolic capture elements 104 accommodate this inflation by also increasing in diameter or otherwise allowing space within their structure for the embolic balloon 102.

The example embolic protection device 100 shown in the figures is illustrated with a total of 4 embolic capture elements 104, however, any number of elements 104 may be used as previously described (e.g., 1, 2, 3, 4, 5, 6, 7, 8, 9, 10 or more). In some examples, the embolic capture elements 104 may be spaced apart at uniform distances from each other or non-uniform distances from each other. For example, the embolic capture elements 104 may have shorter spacing near the distal, proximal, or middle regions of the embolic balloon 102 relative to other regions.

The example embolic protection device 100 is illustrated with embolic capture elements 104 that may be one or more wires forming a coil shape around the embolic balloon 102. This coil shape may allow them to partially coil and uncoil as the embolic balloon 102 is expanded or contracted.

Alternatively, the embolic capture elements 104 may be composed of a plurality of struts connected together to form a web or a plurality of braided wires that create a mesh, both of which surround the embolic balloon 102 similar to the wire coils. While the example of the embolic capture elements 104 forming coils are discussed further below, it should be understood that alternative versions comprising struts or mesh may also be used. Additionally, if a plurality of embolic capture elements 104 are used, different types/structures of embolic capture elements 104 may be used (e.g., any combination of coils, struts, and/or mesh).

Several different expanded shapes of the embolic capture elements 104 are possible. In the example of FIGS. 1-4, the embolic capture elements 104 expands to a conical shape that increases in diameter in a proximal direction (e.g., toward a proximal end of the elongated catheter body 106). FIG. 7 is a cross sectional view taken along the cross section lines in FIG. 2, and illustrates a portion of one of the embolic capture elements 104 adjacent to the embolic balloon 102. As seen in FIG. 7, the one or more wires radially increase in diameter in a proximal direction as it loops around or encircles the embolic balloon 102 to form a conical shape 104A.

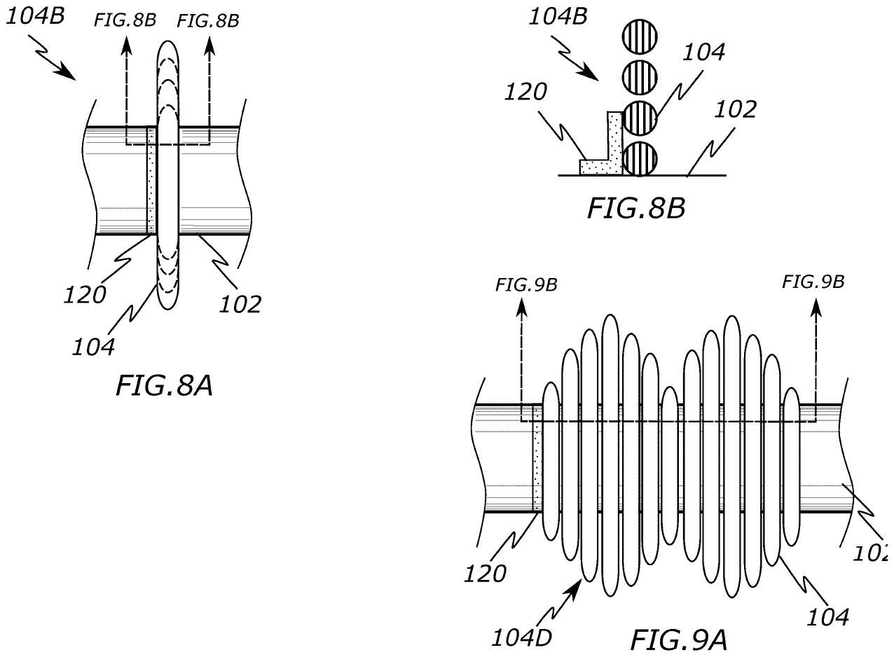

Alternatively, other expanded shapes of the embolic capture elements 104 may be possible. For example, FIG. 8A illustrates a side view of a vertical shape 104B of the embolic capture elements 104 in their expanded configuration. FIG. 8B illustrates a cross sectional view of the vertical shape 104B taken along the cross section lines of FIG. 8A. Each loop of the one or more wires of the embolic capture elements 104 may increase in diameter, generally without leaning or angling either proximally or distally (i.e., generally perpendicular to a longitudinal axis of the embolic protection device 100). Put another way, the vertical shape 104B may form a generally flat disc or flat ring shape around the embolic balloon 102.

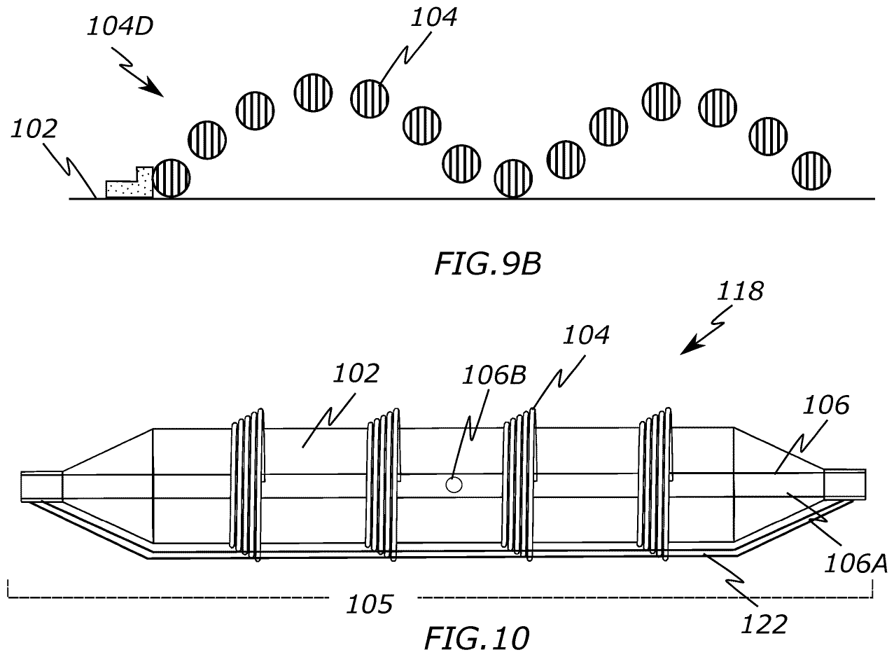

In another example, FIG. 9A illustrates a side view of a wave-like shape 104D of the embolic capture elements 104. FIG. 9B illustrates a cross sectional view of the wave-like shape 104D taken along the cross section lines of FIG. 9A. Each of the loops of the one or more wires may progressively increase in radius and then decrease in radius along a longitudinal length of the embolic balloon 102. The wave-like shape 104D may form several discrete sections or a single continuous wave-like shape along part or all of the length of the embolic balloon 102.

In the example embolic protection device 100, the embolic capture elements 104 may be connected to an outer surface of the embolic balloon 102. For example, an elastomeric band 120 may be connected to an outer surface of the embolic balloon 102 and to a portion of an embolic capture element 104, such as a distal and/or proximal surface of the embolic capture element 104. In other examples, the embolic capture elements 104 may be connected via adhesive or may be embedded within the wall of the embolic balloon 102. In another example, one or more rings may be positioned through portions of the embolic capture element 104. The one or more rings may be attached or partially embedded in the embolic balloon 102 or may be attached at other locations on the embolic protection device 100. The one or more rings may be relatively smaller in diameter, such as eyelets positioned along a side of the embolic balloon 102 or may be positioned around a circumference of the embolic balloon 102. The one or more rings may be composed of metal (e.g., Nitinol) or a polymer, and may be in the form of a complete solid ring, a coil, or a similar shape.

Alternatively, FIG. 10 illustrates an embolic protection device 118 in which the embolic capture elements 104 may be connected via a framework 122 comprising one or more wires or struts that connect to the elongated catheter body 106. In the present example, the framework 122 includes one or a plurality of longitudinal struts that connect at locations on the elongated catheter body 106 proximal and/or distal to the embolic balloon 102, thereby supporting the embolic capture elements 104 separately of the embolic balloon 102.

In any of the shapes described in this specification, the one or more wires may be composed of a shape memory alloy, such as Nitinol, a resilient polymer, or similar material. Any of the shapes of this specification may be configured to have an expanded shape with a diameter that is radially larger than a fully expanded diameter of the embolic balloon 102.

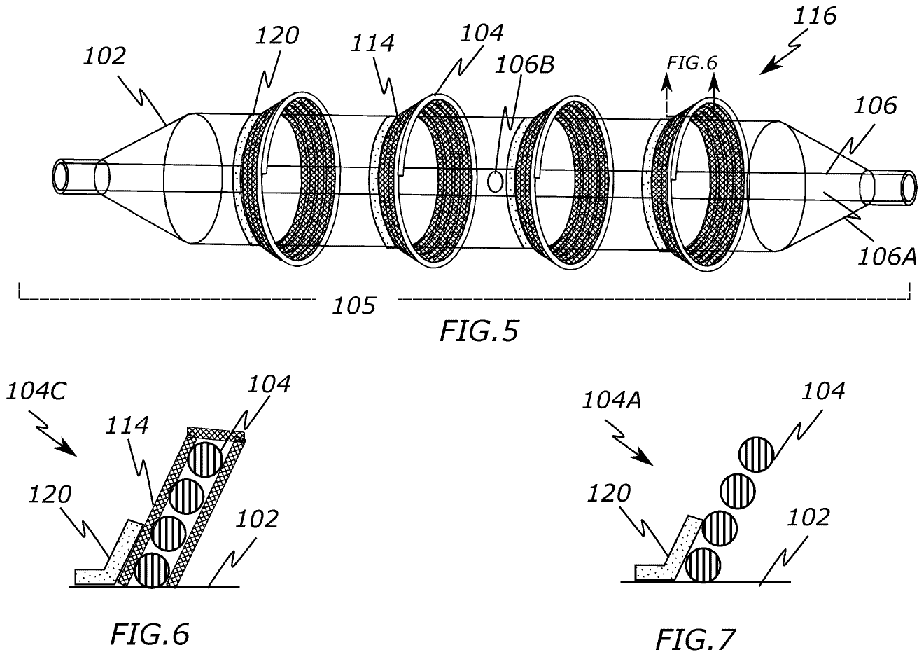

Part or all of the embolic capture elements 104 may optionally include a filter sheet or membrane with pores sized to allow blood flow therethrough but to capture embolic material. For example, FIG. 5 illustrates a side perspective view of an embolic protection device 116 with an embolic capture element 104 comprising one or more wires that form a shape 104C that is generally conical and comprises a membrane 114 connected to the one or more wires. FIG. 6 illustrates a cross sectional view of a portion of the embolic capture elements 104 taken along the cross sectional lines indicated in FIG. 5 and will be discussed concurrently with FIG. 5.

In the example of FIGS. 5 and 6, the membrane 114 is located on a proximal and a distal side of the embolic capture elements 104, and may completely enclose the embolic capture elements 104. However, the membrane 114 may alternatively be located only on a proximal or a distal side of the embolic capture elements 104, or may be interposed or interwoven between the one or more wires/coils of the embolic capture elements 104. The membrane 114 may be connected to the embolic capture elements 104 via adhesives, threads, wires, welding, or similar adhesive techniques. In the example of the one or more wires forming a coil that makes up the embolic capture elements 104, the membrane 114 may provide slack between each loop of the coil to allow for the loops of the coil to become closer or more spaced out, depending on whether they are in the radially expanded configuration or the radially contracted configuration. The membrane 114 may also allow the one or more wires of the embolic capture elements 104 to move relative to the membrane 114 (e.g., by creating a lumen or passage for the one or more wire) so that the wire may partially coil and uncoil, depending on whether it is in the radially expanded configuration or the radially contracted configuration.

The membrane 114 may be a wire mesh, a woven fabric, a porous polymeric material, or similar porous materials. The membrane 114 may have a plurality of pores that allow blood to pass through while substantially preventing embolic material from passing through. For example, the plurality of pores may have a porosity or diameter within an inclusive range of about 0.9 microns to about 1 mm. In one specific example, the material may be ePTFE that is made porous such that blood passes through but not larger emboli.

As previously discussed, the embolic protection device 100 (or any variations discussed in this specification) may also include another treatment component such that, after the embolic balloon 102 is inflated, the other treatment component may be used. Any such further treatment may occur as part of the same embolic protection device 100 or as a separate device deployed separately.

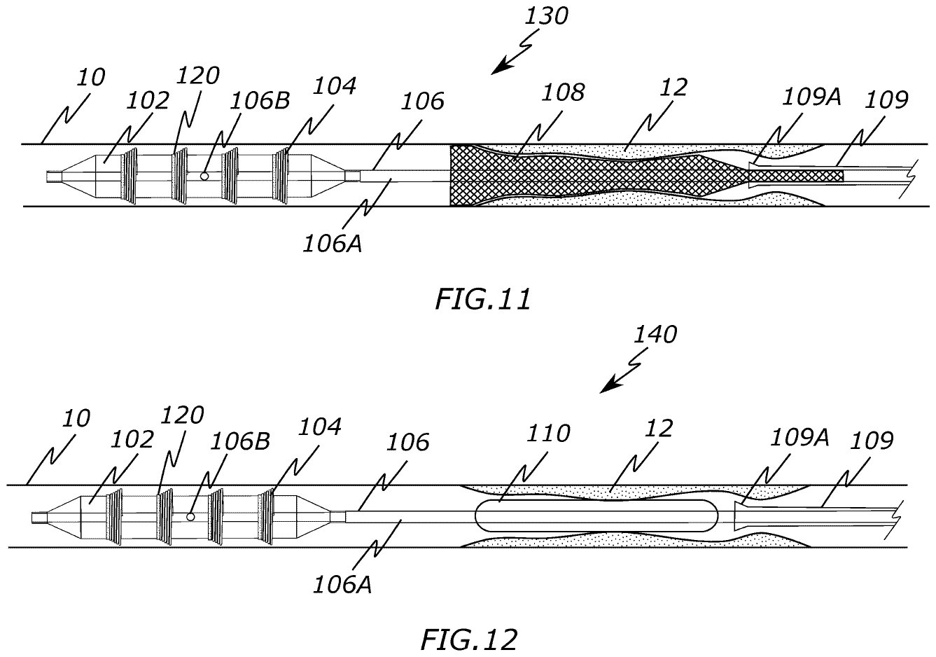

For example, FIG. 11 illustrates a side view of an embolic protection and treatment device 130 that is generally similar to any of the example embolic protection devices described in this specification. However, the elongated catheter body 106 may also include a stent 108 proximal of the embolic balloon 102 that may be deployed over a plaque in a vessel 10 or for other purposes. Alternatively, the stent 108 may be deployed on a separate delivery device.

In operation, a distal end of the embolic protection and treatment device 130 may be positioned at or near a target location in a vessel. An outer sheath 109 may be proximally withdrawn to expose the embolic balloon 102. The embolic balloon 102 may be inflated such that the embolic capture elements 104 are positioned near or in contact with the walls of the vessel 10. The outer sheath 109 may be further proximally retracted to expose the stent 108, allowing it to radially expand against the walls of the vessel 10. Once the stent 108 is deployed, the embolic balloon 102 may be deflated and either proximally pulled into the outer sheath 109 or the outer sheath 109 may be moved distally to cover the embolic balloon 102 and the entire embolic protection and treatment device 130 may be withdrawn. The outer sheath 109 may include a conical or funnel-shaped distal end 109A of its lumen which may help the embolic capture elements 104 to be radially compressed to a diameter sufficient in size to fit within the remaining portion of the outer sheath 109.

In another example, FIG. 12 illustrates a side view of an embolic protection and treatment device 140 that is generally similar to any of the example embolic protection devices described in this specification. However, the elongated catheter body 106 may also include an angioplasty balloon 110 that is located proximally of the embolic balloon 102. The angioplasty balloon 110 may have its own inflation lumen in the catheter body 106 such that it can inflate against a plaque 12 in a vessel 10 to perform angioplasty. Alternatively, the angioplasty balloon 110 may be located on a separate catheter from the embolic protection and treatment device 140.

In operation, a distal end of the embolic protection and treatment device 140 may be positioned at or near a target location in a vessel. An outer sheath 109 may be proximally withdrawn to expose the embolic balloon 102. The embolic balloon 102 may be inflated such that the embolic capture elements 104 are positioned near or in contact with the walls of the vessel 10. The outer sheath 109 may be further proximally retracted to expose the angioplasty balloon 110, allowing it to radially expand against the plaque 12 of the vessel 10. The angioplasty balloon 110 may additionally have a stent 108 positioned over it to allow both angioplasty and stent 108 placement over the plaque 12. Once the angioplasty balloon 110 is deployed, the embolic balloon 102 may be deflated and either proximally pulled into the outer sheath 109 or the outer sheath 109 may be moved distally to cover the embolic balloon 102 and the entire embolic protection and treatment device 130 may be withdrawn. Again, the outer sheath 109 may include a conical or funnel-shaped distal end 109A of its lumen which may help the embolic capture elements 104 to be radially compressed to a diameter sufficient in size to fit within the remaining portion of the outer sheath 109. In one example, the diameter of the conical or funnel-shaped distal end 109A may be substantially the same or have a larger diameter than the diameter of the embolic capture elements 104 in a compressed or expanded configuration.

While both the stent 108 and angioplasty balloon 110 are illustrated as being located proximal of the embolic balloon 102, it is also possible for the stent 108 and the angioplasty balloon 110 to be positioned distally of the embolic balloon 102, depending on the direction of blood flow relative to the direction of access of a target area.

### Claim Bank

Clause 1. An embolic protection device, comprising: an elongated catheter body; an embolic balloon located at a distal portion of the elongated catheter body; and, one or more embolic capture elements that are each positioned around the embolic balloon; wherein the one or more embolic capture elements extend radially outward from the embolic balloon when the embolic balloon is inflated to capture embolic material.

Clause 2. The embolic protection device of clause 1, wherein the one or more embolic capture elements comprise 2, 3, 4, 5, 6, 7, 8, 9, or 10 embolic capture elements.

Clause 3. The embolic protection device of clause 2, wherein embolic capture elements have a uniform spacing or a non-uniform spacing.

Clause 4. The embolic protection device of clause 1, wherein the one or more embolic capture elements form a conical shape opening towards a proximal end of the elongated catheter body.

Clause 5. The embolic protection device of clause 1, wherein the one or more embolic capture elements form a flat disc shape around the embolic balloon.

Clause 6. The embolic protection device of clause 1, wherein the one or more embolic capture elements form a wave-like shape that progressively increases and decreases in radius along a longitudinal length of the embolic balloon.

Clause 7. The embolic protection device of clause 1, wherein the one or more embolic capture elements comprise a wire forming a coil shape around the embolic balloon.

Clause 8. The embolic protection device of clause 1, wherein the one or more embolic capture elements comprise a plurality of struts connected together to form a web surrounding the embolic balloon.

Clause 9. The embolic protection device of clause 1, wherein the one or more embolic capture elements comprise a plurality of braided wires that form a mesh surrounding the embolic balloon.

Clause 10. The embolic protection device of clause 1, wherein the one or more embolic capture elements are connected to an outer surface of the embolic balloon via an elastomeric band or metal coil ring.

Clause 11. The embolic protection device of clause 1, wherein the one or more embolic capture elements are embedded within a wall of the embolic balloon.

Clause 12. The embolic protection device of clause 1, further comprising a framework connected to the one or more embolic capture elements and to the elongated catheter body, supporting the embolic capture elements separately of the embolic balloon.

Clause 13. The embolic protection device of clause 12, wherein the framework is connected to the elongated catheter body distally, proximally, or both distally and proximally of the embolic balloon.

Clause 14. The embolic protection device of clause 1, further comprising a stent that is deployable from the elongated catheter body.

Clause 15. The embolic protection device of clause 1, further comprising an angioplasty balloon connected on the elongated catheter body.

Clause 16. The embolic protection device of clause 1, further comprising a filter material having a plurality of pores allowing blood to pass therethrough.

Clause 17. An embolic protection device, comprising: an elongated catheter body; an embolic balloon located at a distal portion of the elongated catheter body; the embolic balloon having an inflated state and a deflated state; and, one or more embolic capture elements that are each positioned around the embolic balloon; wherein the one or more embolic capture elements extend radially away from the embolic balloon when the embolic balloon is in the inflated state.

Clause 18. The embolic protection device of clause 17, wherein the one or more embolic capture elements each comprise one or more wires forming a coil surrounding the embolic balloon.

Clause 19. The embolic protection device of clause 18, wherein each coil further comprises a blood permeable membrane connected to a proximal side or a distal side of the coil.

Clause 20. An embolic protection device, comprising: an elongated catheter body; an embolic balloon located at a distal portion of the elongated catheter body; and, one or more embolic capture means for capturing embolic material; wherein each are each positioned around the embolic balloon.

Clause 21. A method of using an embolic protection device, comprising: advancing a distal portion of an elongated catheter body near a treatment location; inflating an embolic balloon located at a distal portion of the elongated catheter body such that one or more embolic capture elements extend radially from the embolic balloon; and, performing a treatment at the treatment location such that embolic material is captured by the embolic capture elements.

Clause 22. The method of clause 21, wherein performing the treatment further comprises inflating an angioplasty balloon and/or delivery a stent.

1. An embolic protection device, comprising:
an elongated catheter body;
an embolic balloon located at a distal portion of the elongated catheter body; and,
one or more embolic capture elements that are each positioned around the embolic balloon;
wherein the one or more embolic capture elements extend outward from the embolic balloon when the embolic balloon is at an inflated configuration; and,
wherein the one or more embolic capture elements each comprise one or more wires coiled at least once around an outside of the embolic balloon.

2. The embolic protection device of claim 1, wherein the one or more embolic capture elements comprise 2 or more embolic capture elements.

3. The embolic protection device of claim 2, wherein the one or more embolic capture elements comprise a plurality of embolic capture elements that have a uniform spacing or a non-uniform spacing relative to each other.

4. The embolic protection device of claim 1, wherein the one or more embolic capture elements form a conical shape opening towards a proximal end of the elongated catheter body.

5. The embolic protection device of claim 1, wherein the one or more embolic capture elements form a flat disc shape around the embolic balloon.

6. The embolic protection device of claim 1, wherein the one or more embolic capture elements form a wave-like shape that progressively increases and decreases in radius along a longitudinal length of the embolic balloon.

7. The embolic protection device of claim 1, wherein the one or more wires are coiled a plurality of times around the embolic balloon.

8. The embolic protection device of claim 1, wherein the one or more embolic capture elements comprise a plurality of struts connected together to form a web surrounding the embolic balloon.

9. The embolic protection device of claim 1, wherein the one or more embolic capture elements comprise a plurality of braided wires that form a mesh surrounding the embolic balloon.

10. The embolic protection device of claim 1, wherein the one or more embolic capture elements are connected to an outer surface of the embolic balloon via an elastomeric band or ring.

11. The embolic protection device of claim 1, wherein the one or more embolic capture elements are embedded within a wall of the embolic balloon.

12. The embolic protection device of claim 1, further comprising a framework connected to the one or more embolic capture elements and to the elongated catheter body, supporting the one or more embolic capture elements separately of the embolic balloon.

13. The embolic protection device of claim 12, wherein the framework is connected to the elongated catheter body distally, proximally, or both distally and proximally of the embolic balloon.

14. The embolic protection device of claim 1, further comprising a stent that is deployable from the elongated catheter body.

15. The embolic protection device of claim 1, further comprising an angioplasty balloon connected on the elongated catheter body.

16. The embolic protection device of claim 1, further comprising a filter material having a plurality of pores allowing blood to pass therethrough.

17. An embolic protection device, comprising:
an elongated catheter body;
an embolic balloon located at a distal portion of the elongated catheter body; the embolic balloon having an inflated state and a deflated state; and,
one or more embolic capture elements that are each positioned around the embolic balloon;
wherein the one or more embolic capture elements extend radially away from the embolic balloon when the embolic balloon is in the inflated state; and,
wherein the one or more embolic capture elements each comprise one or more wires coiled at least once around an outside of the embolic balloon.

18. The embolic protection device of claim 17, wherein the one or more embolic capture elements comprise a plurality of embolic capture elements.

19. The embolic protection device of claim 17, wherein the coil further comprises a blood permeable membrane connected to a proximal side or a distal side of the coil.

20. An embolic protection device, comprising:
an elongated catheter body;
an embolic balloon located at a distal portion of the elongated catheter body; and,
one or more embolic capture means for capturing embolic material; wherein each are each positioned around the embolic balloon and comprise one or more wires coiled at least once around an outside of the embolic balloon.

21. A method of using an embolic protection device, comprising:
advancing a distal portion of an elongated catheter body near a treatment location;
inflating an embolic balloon located at a distal portion of the elongated catheter body such that one or more embolic capture elements extend radially from the embolic balloon; wherein the one or more embolic capture elements each comprise one or more wires coiled at least once around an outside of the embolic balloon; and,
performing a treatment at the treatment location such that embolic material is captured by the one or more embolic capture elements.

22. The method of claim 21, wherein performing the treatment further comprises inflating an angioplasty balloon and/or delivering a stent.
